(19) United States
(12) Patent Application Publication
(10) Pub. No.: US 2024/0049970 A1
(43) Pub. Date: Feb. 15, 2024

(54) SYSTEMS, METHODS AND APPARATUS FOR GENERATING BLOOD PRESSURE ESTIMATIONS USING REAL-TIME PHOTOPLETHYSMOGRAPHY DATA

(71) Applicant: SunTech Medical, Inc., Morrisville, NC (US)

(72) Inventors: Paul Motoi MATSUMURA, Cary, NC (US); Julian MULLANEY, Raleigh, NC (US); Robert Lee SWEITZER, Apex, NC (US); Namita LOKARE, Raleigh, NC (US); Tushar Dilip TANK, Raleigh, NC (US); Steven Francis LEBOEUF, Raleigh, NC (US)

(21) Appl. No.: 18/256,013

(22) PCT Filed: Dec. 23, 2021
(86) PCT No.: PCT/US2021/065113

§ 371 Date: Jun. 5, 2023

Related U.S. Application Data
(60) Provisional application No. 63/132,216, filed on Dec. 30, 2020.

(51) Int. Cl. A61B 5/021; A61B 5/00; A61B 5/026; A61B 5/022

(52) U.S. Cl. CPC A61B 5/02116; A61B 5/7267; A61B 5/0261; A61B 5/7275; A61B 5/7282; A61B 5/746; A61B 5/02241; A61B 5/7278; A61B 2560/02; A61B 2560/0462; A61B 2562/0233

(57) ABSTRACT

A blood pressure monitoring system includes a blood pressure monitoring device configured to obtain a blood pressure measurement from a subject, a PPG sensor configured to obtain PPG data from the subject, and at least one processor configured to generate a blood pressure estimation for the subject via an adaptive predictive model using real-time PPG data from the PPG sensor, determine whether the generated blood pressure estimation is above or below a threshold, and send an alert to a remote device in response to determining that the generated blood pressure estimation is above or below the threshold. The at least one processor may also be configured to request a blood pressure measurement from the blood pressure monitoring device in response to determining that the generated blood pressure estimation is above or below the threshold, and update the one or more parameters of the adaptive predictive model in real-time.

### RELATED APPLICATION

This application claims the benefit of and priority to U.S. Provisional Patent Application No. 63/132,216 filed Dec. 30, 2020, the disclosure of which is incorporated herein by reference as if set forth in its entirety.

### FIELD OF THE INVENTION

The present invention relates generally to wearable devices, and more particularly to wearable biometric sensor technology for physiological monitoring for medical, health, and fitness applications.

### BACKGROUND OF THE INVENTION

Noninvasive continuous real-time blood pressure (BP) estimation technology exists in today's marketplace in the form of the “volume clamp” method, a technology in which a photoplethysmography (PPG) sensor and clamp are placed around a subject's finger to actively monitor the blood flow and the clamp pressure is adjusted to maintain a constant blood flow through the finger during each pulse. This clamp pressure is directly related to the subject's blood pressure and, following a series of calibrations against an automated upper arm (brachial artery) cuff-based BP monitor, the volume clamp method can (in some limited circumstances) be applied towards roughly estimating subject blood pressure continuously without requiring that an arterial line pressure sensor be invasively inserted within the subject. Examples of BP monitoring systems utilizing the volume clamp method are those provided by CNSystems, Edwards Lifesciences and Finapres.

Although the volume clamp method is currently the commercial workhorse of noninvasive continuous blood pressure monitoring, the inventors have discovered that this method suffers from several limitations: 1) a mechanical finger cuff is required, making the method unsuitable for ambulatory use in everyday life activities, 2) the transfer function between the finger blood flow, clamp pressure, and brachial artery pressure can change over a short time, leading to unpredictable calibration drift, 3) physiological extrema, such as fat fingers or hardened arteries, can pose an inherent limit on the ability to accurately calibrate the clamp pressure to the brachial artery pressure, 4) the system is quite sensitive to motion artifacts, reducing the utility to stationary use cases only, 5) because continuously active mechanical parts are required, the solution is not suitable for truly wearable, free-living use cases where battery life is a precious resource, 6) BP estimations are insufficiently accurate in periods when the brachial arterial blood pressure rapidly increases or decreases, and 7) numerous other limitations that continue to prevent the method from serving as a viable alternative to the arterial line for continuous BP measurements.

### SUMMARY

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, a blood pressure monitoring system includes a blood pressure monitoring device configured to obtain a blood pressure measurement from a subject, an arterial pulse wave sensor configured to obtain arterial pulse wave data from the subject, and at least one processor configured to generate a blood pressure estimation for the subject via an adaptive predictive model (e.g., a regression model, a machine learning model, a classifier model, etc.) using real-time arterial pulse wave data from the arterial pulse wave sensor. The at least one processor is further configured to receive a real-time blood pressure measurement from the blood pressure monitoring device, and in response to receiving the real-time blood pressure measurement, update one or more parameters of the adaptive predictive model in real-time to improve blood pressure estimation accuracy of the adaptive predictive model. The at least one processor may also be configured to determine whether the generated blood pressure estimation is above or below one or more thresholds, and in response to determining that the generated blood pressure estimation is above or below one or more thresholds, update the one or more parameters of the adaptive predictive model in real-time. The at least one processor is configured to receive subsequent real-time blood pressure measurements from the blood pressure monitoring device, and in response to receiving the real-time blood pressure measurements, update one or more parameters of the adaptive predictive model in real-time. The at least one processor may also be configured to send an alert to a remote device in response to determining that the generated blood pressure estimation is above or below a threshold. The at least one processor may also be configured to request a blood pressure measurement from the blood pressure monitoring device in response to determining that the generated blood pressure estimation is above or below one or more thresholds.

In some embodiments, the arterial pulse wave sensor is a PPG sensor. In some embodiments the blood pressure monitoring device is an inflatable cuff configured to be attached to a limb or digit of a subject.

According to other embodiments of the present invention, a wearable device includes an automated inflatable cuff configured to be attached to a limb or digit of a subject, an arterial pulse wave sensor, and at least one processor. The at least one processor is configured to generate a blood pressure estimation for the subject via an adaptive predictive model (e.g., a regression model, a machine learning model, a classifier model, etc.) using real-time arterial pulse wave data from the arterial pulse wave sensor, receive a real-time blood pressure measurement from the cuff, and in response to receiving the real-time blood pressure measurement, update one or more parameters of the adaptive predictive model in real-time to improve blood pressure estimation accuracy of the adaptive predictive model. The at least one processor may also be configured to determine whether the generated blood pressure estimation is above or below one or more thresholds, and in response to determining that the generated blood pressure estimation is above or below one or more thresholds, update the one or more parameters of the adaptive predictive model in real-time. The at least one processor is further configured to, in response to receiving one or more subsequent real-time blood pressure measurements, update the one or more parameters of the adaptive predictive model in real-time. The at least one processor may also be configured to send an alert to a remote device in response to determining that the generated blood pressure estimation is above or below a threshold. The at least one processor may also be configured to request a blood pressure measurement from the cuff in response to determining that the generated blood pressure estimation is above or below one or more thresholds.

According to other embodiments of the present invention, a blood pressure monitoring system includes a blood pressure monitoring device configured to obtain a blood pressure measurement from a subject, a PPG sensor configured to obtain PPG data from the subject, and at least one processor configured to generate a blood pressure estimation for the subject via an adaptive predictive model (e.g., a regression model, a machine learning model, a classifier model, etc.) using real-time PPG data from the PPG sensor, determine whether the generated blood pressure estimation is above or below one or more thresholds, and send an alert to a remote device in response to determining that the generated blood pressure estimation is above or below a threshold. The at least one processor may also be configured to request a blood pressure measurement from the blood pressure monitoring device in response to determining that the generated blood pressure estimation is above or below one or more thresholds, and update the one or more parameters of the adaptive predictive model in real-time.

According to other embodiments of the present invention, a method of determining blood pressure variability for a subject includes the following steps performed by at least one processor: receiving, from a blood pressure monitoring device attached to the subject (e.g., an inflatable cuff configured to be attached to a limb or digit of a subject, etc.), a blood pressure measurement at a first time; receiving, from the blood pressure monitoring device, a blood pressure measurement at a second time; receiving, from a photoplethysmography (PPG) sensor attached to the subject, PPG waveform data during a time period between the first time and the second time; and generating an estimation of blood pressure variability during the time period based on PPG waveform fluctuations identified during the time period. The estimation of blood pressure variability may include an estimation of systolic blood pressure variation and/or an estimation of diastolic blood pressure variation. In some embodiments, the estimation of blood pressure variability may include an estimation of mean blood pressure variation.

According to other embodiments of the present invention, a blood pressure monitoring system includes a blood pressure monitoring device configured to obtain a blood pressure measurement from a subject (e.g., an inflatable cuff configured to be attached to a limb or digit of a subject, etc.), a photoplethysmography (PPG) sensor configured to obtain PPG waveform data from the subject, and at least one processor. The at least one processor is configured to receive a blood pressure measurement at a first time from the blood pressure monitoring device, receive a blood pressure measurement at a second time from the blood pressure monitoring device, receive PPG waveform data from the PPG sensor during a time period between the first time and the second time, and generate an estimation of blood pressure variability during the time period based on PPG waveform fluctuations identified during the time period. The estimation of blood pressure variability may include an estimation of systolic blood pressure variation and/or an estimation of diastolic blood pressure variation. In some embodiments, the estimation of blood pressure variability may include an estimation of mean blood pressure variation.

According to other embodiments of the present invention, a method of determining blood pressure variability for a subject includes the following steps performed by at least one processor: receiving a blood pressure measurement from a blood pressure monitoring device attached to the subject (e.g., an inflatable cuff configured to be attached to a limb or digit of a subject, etc.) and photoplethysmography (PPG) waveform data from a PPG sensor attached to the subject; receiving PPG waveform data from the PPG sensor during a time period after the first time; and generating an estimation of blood pressure variability during the time period based on PPG waveform fluctuations identified during the time period. The estimation of blood pressure variability may include an estimation of systolic blood pressure variation and/or an estimation of diastolic blood pressure variation. In some embodiments, the estimation of blood pressure variability may include an estimation of mean blood pressure variation.

According to other embodiments of the present invention, a blood pressure monitoring system includes a blood pressure monitoring device configured to obtain a blood pressure measurement from a subject (e.g., an inflatable cuff configured to be attached to a limb or digit of a subject, etc.), a photoplethysmography (PPG) sensor configured to obtain PPG waveform data from the subject, and at least one processor. The at least one processor is configured to receive a blood pressure measurement from the blood pressure monitoring device and photoplethysmography (PPG) waveform data from the PPG sensor, receive PPG waveform data from the PPG sensor during a time period after the first time, and generate an estimation of blood pressure variability during the time period based on PPG waveform fluctuations identified during the time period. The estimation of blood pressure variability may include an estimation of systolic blood pressure variation and/or an estimation of diastolic blood pressure variation. In some embodiments, the estimation of blood pressure variability may include an estimation of mean blood pressure variation.

According to other embodiments of the present invention, a method of determining blood pressure variability for a subject, the method comprising the following steps performed by at least one processor: receiving and storing blood pressure measurements from a blood pressure monitoring device attached to the subject over a period of time; receiving and storing photoplethysmography (PPG) data from a PPG sensor attached to the subject over the period of time; and processing the stored blood pressure measurements and the stored PPG data to generate an estimation of blood pressure variability during the time period. In some embodiments, the PPG data includes PPG waveform data, and the at least one processor is further configured to process the stored blood pressure measurements and the stored PPG data to generate an estimation of blood pressure variability during the time period based on PPG waveform fluctuations identified during the time period.

According to other embodiments of the present invention, a blood pressure monitoring method comprises the following steps performed by at least one processor: receiving real-time arterial pulse wave data from an arterial pulse sensor attached to a subject over a period of time; generating blood pressure estimations for the subject via an adaptive predictive model using the arterial pulse wave data; and generating an estimation of blood pressure variability during the period of time. The method may further include the following steps performed by at least one processor: receiving real-time blood pressure measurements from a blood pressure monitoring device attached to the subject over the period of time; and using the real-time blood pressure measurements to update one or more parameters of the adaptive predictive model in real-time to improve blood pressure estimation accuracy of the adaptive predictive model.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 illustrates a computational system for generating BP estimations, according to some embodiments of the present invention.

FIGS. 2-4 are flowcharts of methods of generating BP estimations, according to some embodiments of the present invention.

FIG. 5 illustrates exemplary wearable devices that may be utilized in accordance with embodiments of the present invention.

FIG. 6 illustrates a sliding window of time that may be utilized to receive PPG data and blood pressure measurement data, according to some embodiments of the present invention.

FIG. 7 is a block diagram illustrating operations for updating one or more parameters of an adaptive predictive model, according to some embodiments of the present invention.

FIG. 8 illustrates an adaptive predictive model, according to some embodiments of the present invention.

FIG. 9 is a data plot collected from a subject wearing a blood pressure cuff and a PPG sensor, and illustrating the collection of real-time BP measurement data and real-time PPG-BP estimations, according to some embodiments of the present invention.

FIG. 10 is a graphic output of estimated blood pressure and actual blood pressure for a subject over a time period and illustrating improvement of blood pressure estimation over time via augmentation.

FIG. 11 illustrates tables comparing volume clamp BP estimations with PPG-BP estimates according to embodiments of the present invention, in terms of accuracy with respect to actual BP measurements.

FIG. 12 is a block diagram that illustrates details of an example processor and memory that may be used in accordance with various embodiments of the present invention.

FIGS. 13-16 are flowcharts of methods of generating estimations of BP variability, according to some embodiments of the present invention.

### DETAILED DESCRIPTION

The term “subject”, as used herein, typically refers to a human being in context of the invention description. However, in context of the invention, a subject may also be a living creature that is not a human being.

The term “biometric” generally refers to a metric for a subject generated by processing physiological (i.e., biological) information from the subject. Nonlimiting examples of biometrics may include: heart rate (HR), heart rate variability (HRV), RR-interval, respiration rate, weight, height, sex, physiological status, overall health status, disease conditions, injury status, blood pressure, arterial stiffness, cardiovascular fitness, VO₂max, gas exchange analysis metrics, blood analyte levels fluid metabolite levels, and the like.

The terms “biometric” and “physiological metric”, as used herein are interchangeable.

The term “real-time” is used herein to describe a process that requires a period of time that appears substantially real-time to a human individual. Thus, the term “real-time” is used interchangeably to mean “near real-time” or “quasi-real-time”. Namely, a “real-time” process may refer to an “instantaneous process” but may also refer to a process that generates an output within a short enough processing time to (in effect) be as useful as an instantaneous process (in context of a particular use case). For example, in practicality, a process that requires several seconds or minutes to generate a blood pressure metric for a subject may be considered to be a real time process, as used herein, even though blood pressure may be changing each second, as the use case may involve a sedentary state for the subject where subtle changes in blood pressure may be insignificant and averaged out.

The terms “respiration rate” and “breathing rate”, as used herein, are interchangeable.

The terms “heart rate” and “pulse rate”, as used herein, are interchangeable.

The term “system”, as used herein, refers to a collection of physical and/or computational materials that may be unified by a common function.

The terms “motion sensor”, as used herein, refers to a sensor configured to sense motion information (e.g., from a subject). Nonlimiting examples of motion sensors may comprise: single- or multi-axis inertial sensors (such as accelerometers, gyroscopes, MEMS motion sensors, and the like), optical scatter sensors, blocked channel sensors, and the like.

The term “photoplethysmography” (PPG), as used herein, refers to a method of generating physiological information from PPG waveforms collected via a PPG sensor.

The term “PPG waveform”, as used herein, refers to physiological waveform data resulting from a temporal modulation of photon flux through physiological material.

The term “PPG sensor”, as used herein, refers to a sensor configured to sense photons and generate PPG waveform data. A typical PPG sensor may comprise an optical sensor configured to sense photons in the optical spectrum (i.e., an electromagnetic wavelength range of ˜10 nm to 103 μm, or electromagnetic frequencies in the range from ˜300 GHz to 3000 THz). Nonlimiting examples of optical sensors may comprise inorganic and/or organic photodetectors (such as photoconductors, photodiodes, phototransistors, phototransducers, and the like), reverse-biased light-emitting diodes (LEDs) or other reverse-biased optical emitters, imaging sensors, photodetector arrays, and the like. Additionally, a typical PPG sensor may also comprise a photon (photonic) emitter to generate a photon flux through a physiological pathway. However, in some cases, ambient photons or photons from another source (that is not part of the PPG sensor) may be used to generate photons. Typical PPG sensors may comprise photon emitters that are optical emitters, such as inorganic and/or organic light-emitting diodes (LEDs), laser diodes (LDs), microplasma sources, or the like. PPG sensors may also comprise a motion sensor for the purposes of generating subject activity data and/or providing a noise reference for attenuating motion artifacts in PPG waveform data.

The terms “sensor”, “sensing element”, and “sensor module”, as used herein, are interchangeable and refer to a sensor element or group of sensor elements that may be utilized to sense information, such as information (e.g., physiological information, body motion, etc.) from the body of a subject and/or environmental information in a vicinity of the subject. A sensor/sensing element/sensor module may comprise one or more of the following: a detector element, an emitter element, a processing element, optics, or optomechanics, sensor mechanics, mechanical support, supporting circuitry, and the like. Both a single sensor element and a collection of sensor elements may be considered a sensor, a sensing element, or a sensor module. A sensor/sensing element/sensor module may be configured to both sense information and process that information into one or more metrics.

As used herein, the term “processor” broadly refers to a signal processing circuit or computing system, or a computational method, which may be localized and/or distributed. For example, a localized signal processing circuit may comprise one or more signal processing circuits or processing methods localized to a general location, such as to a wearable blood pressure monitoring device. Examples of such devices may comprise, but are not limited to, an earpiece, a headpiece, a finger clip, a toe clip, a limb band (such as an arm band or leg band), an ankle band, a wrist band, a digit (e.g., finger or toe) band, a nose band, a sensor patch, jewelry, a patch, apparel (clothing) or the like. Examples of a distributed processing circuit include “the cloud,” the internet, a remote database, a remote processor computer, a plurality of remote processing circuits or computers in communication with each other, etc., or processing methods distributed among one or more of these elements. The difference between distributed and localized processing circuits is that a distributed processing circuit may include delocalized elements, whereas a localized processing circuit may work independently of a distributed processing system. Microprocessors, microcontrollers, or digital signal processing circuits represent a few non-limiting examples of signal processing circuits that may be found in a localized and/or distributed system.

The terms “mobile application”, “mobile app” and “app”, as used herein, are interchangeable and refer to a software program that can run on a computing apparatus, such as a mobile phone, digital computer, smartphone, database, cloud server, processor, wearable device, or the like.

The term “health”, as used herein, is broadly construed to relate to the physiological status of an organism or of a physiological element or process of an organism. For example, cardiovascular health may refer to the overall condition of the cardiovascular system, and a cardiovascular health assessment may refer to an estimate of blood pressure, VO₂max, cardiac efficiency, heart rate recovery, arterial blockage, arrhythmia, atrial fibrillation, or the like. A “fitness” assessment is a subset of a health assessment, where the fitness assessment refers to how one's health affects one's performance at an activity. For example, a VO₂max test can be used to provide a health assessment of one's mortality or a fitness assessment of one's ability to utilize oxygen during an exercise.

The term “blood pressure”, as used herein, refers to a measurement or estimate of the pressure associated with blood flow of a person, such as a diastolic blood pressure, a systolic blood pressure, a mean arterial pressure, or the like. The blood pressure may be with reference to any location on the body where blood vessels and blood flow exists (i.e., brachial, thoracic, subclavian, femoral, tibial, radial, carotid, and the like). The term “blood pressure” is abbreviated as “BP” throughout this document.

As used herein, any device or system is considered to be remote to another device or system as long as there is no physical connection between them. As a point of clarity, the term “remote” does not necessarily mean that a remote device is a wireless device or that it is a long distance away from a device in communication therewith. For example, in some cases, two devices may be considered remote devices with respect to each other even if there is a physical connection between them. In this case, the term “remote” is intended to reference a device or system that is distinct from another device or system or that is not substantially reliant on another device or system for core functionality. For example, a computer wired to a wearable device may be considered a remote device, as the two devices are distinct and/or not substantially reliant on each other for core functionality.

The terms “sampling frequency”, “signal analysis frequency”, and “signal sampling rate”, as used herein, are interchangeable and refer to the number of samples per second (or per other time unit) taken from a continuous sensor or sensing element (for example, the sampling rate of the thermopile output in a tympanic temperature sensor or the sampling rate of the PPG signal from a PPG sensor).

It should be noted that “algorithm” and “circuit” are referred to herein. An algorithm refers to a computational instruction set, such as an instruction set with sequential steps and logic, that may be in memory whereas a circuit refers to electronic components and/or traces that may implement such logic operations in the digital and/or analog domain.

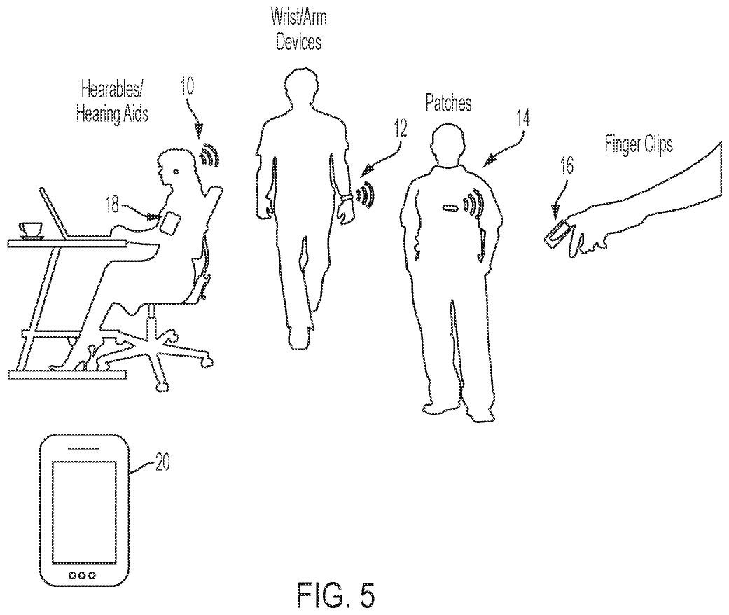

To address these limitations, methods and apparatus according to the present invention provide for continuously generating blood pressure estimates via a real-time adaptive predictive model. These methods and apparatus leverage continuous PPG measurements from a subject, combined with at least one BP measurement from a subject, to update, in real-time, a predictive model for that subject that is more accurate in estimating BP for that subject (than prior to the update). The methods of the present invention may be implemented in a computational system that is configured to receive the PPG and BP data and process this data to improve estimation accuracy. Namely, the model may be configured to generate a BP estimate for a given set of PPG input features, such that the BP estimate is a function of the PPG features, and the parameters of the model may be updated over time as recurring BP measurements (e.g., from a cuff-based BP monitor) are processed to improve the error of the model. In some cases, the computational system may be worn as an ear-worn device (e.g., hearables/hearing aids) 10, as a limb-worn (e.g., wrist, arm, leg) device 12, as a patch 14, as a finger clip 16, as illustrated in FIG. 5.

These wearable PPG devices 12-16 may be in communication (e.g., electrical, optical, or wireless) with a blood pressure monitoring device, such as a blood pressure cuff 18 (such as that shown on the arm of the subject wearing the PPG earpiece 12 in FIG. 5). Alternatively, the blood pressure monitoring device may be another device. Just one of many additional examples would be a standoff device, such an electromagnetic wavelength doppler-based detection system or an imaging system (i.e., a camera). Other blood pressure monitoring devices may be used, as there are many known to those skilled in the art (ultrasound, arterial line, etc.). In another embodiment, the PPG measurements and BP measurements are received from the same device which is configured to measure both PPG and BP readings. One particular example of such a device comprises a cuff-based BP monitor having an integrated PPG sensor.

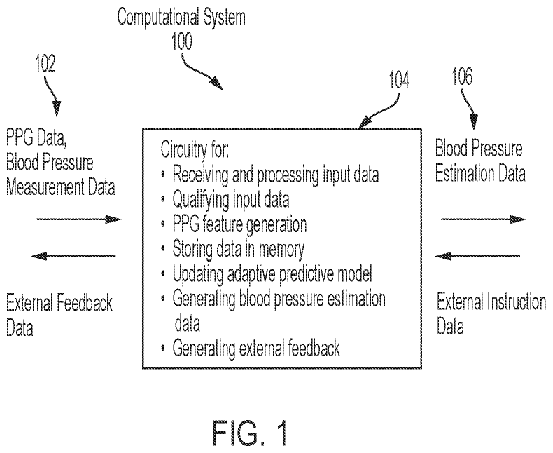

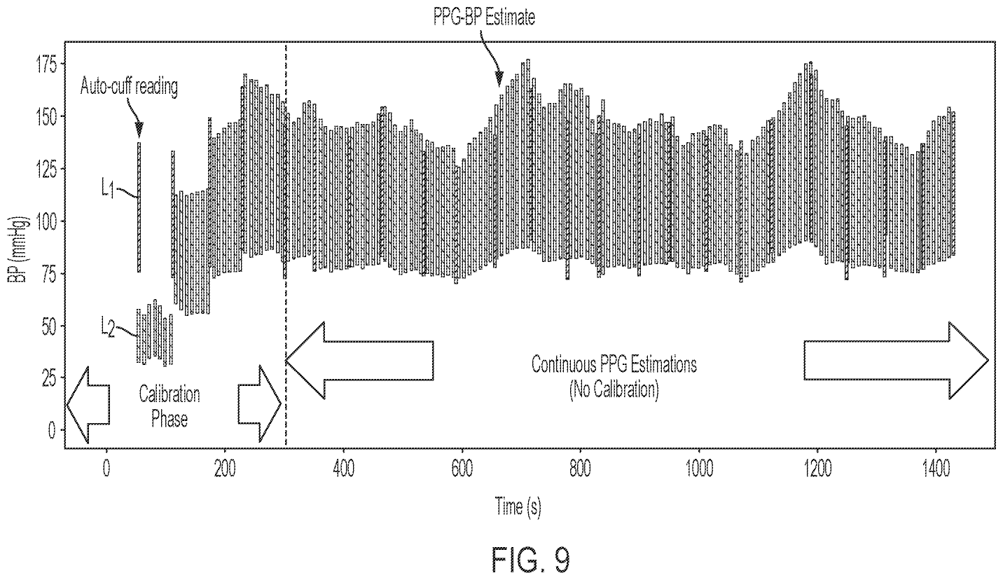

In some embodiments of the present invention, referred to as an adaptation process, a plurality of BP measurements from a cuff-based BP monitor 18 or other BP monitoring device and PPG measurements are processed together to improve the accuracy of the BP estimation. Once the model has been autonomously optimized for the subject, via a computational system (e.g., 100, FIG. 1) processing a plurality of BP measurements and PPG measurements collected as a temporal sequence, the blood pressure measurement device 18 (e.g., a cuff-based BP monitor) may no longer be needed, such that continuous PPG-based BP estimations may be generated in real-time via the updated model. In such case, this period of adaption may behave as a long-term calibration, which may be occasionally re-calibrated a few times of the day, week, month, or year with each new BP measurement (as shown in FIG. 9). Alternatively, BP measurements may be received and processed routinely, referred to as an augmentation process, such that the adaptive predictive model may be continuously augmented over time based on updated BP measurements (such as those taken from an automated cuff-based BP monitor). In augmentation, updating an adaptive predictive model according to embodiments of the present invention may be repeated continuously, several times an hour, with each new BP update.

Referring to FIG. 9, an example of an embodiment of the present invention utilizing real data collected from a human subject in a biometrics laboratory is illustrated. A human subject was wearing an automated cuff-based BP monitor (at the brachial artery) and also wearing an ear PPG sensor, an arm PPG sensor, and a wrist PPG sensor (although only ear-PPG data is presented in FIG. 9 for simplicity). To compare the present invention to the volume clamp method, the subject was also wearing a volume-clamp device on the index finger of the arm where the cuff-based BP monitor was located. The measurement sequence involved periods of subject rest followed by periods of subject activity. Namely, in order to increase the BP of the subject, the subject was asked to push against a stationary barrier with their legs for several seconds (an isometric leg press) while BP and PPG measurements were underway.

Then to decrease BP, the subject was asked to relax by terminating the isometric leg press. BP measurements from the cuff-based BP monitor (presented as a thick vertical line L₁, with the top point of the line L₁ representing the subject systolic BP and the bottom point of the line L₁ representing the subject diastolic BP) were received every 60-to-90 seconds and processed (by a computational system). During an initial calibration phase of approximately 300 seconds, multiple values from the cuff-based BP monitor were processed along with multiple PPG readings to generate multiple PPG estimates (presented as a thin vertical line L2, in the same formalism as the cuff-based readings). However, these estimates were not reported to the user, as the parameters of the adaptive predictive model were updated during this calibration phase to increase model accuracy such that it would be equivalent to that of the cuff-based BP monitor by the end of the calibration phase.

Following the calibration phase, continuous BP estimates were generated without updating model parameters for each new BP measurement. Rather, the remaining cuff-based BP measurements are shown along with PPG estimations simply to note the excellent tracking between the PPG model estimates and the cuff-based measurements. It should be noted that although the PPG estimates shown in FIG. 9 are from the ear PPG sensor only, it was discovered that equivalent performance can be realized via the wrist PPG sensor and the arm PPG sensor. However, as the blood pressure cuff inflates and deflates, there is a period where occlusion can affect the blood flow (when the wrist and/or arm sensors are worn on the same arm as the cuff), such that meaningful PPG-BP estimations are not viable during the cuff-based BP monitor measurement period.

The test sequence of FIG. 9 was repeated on several subjects, and the performance of the PPG-BP estimation (also called the estimated BP measurement, or PPG-eBP) and the volume clamp device, as compared to the cuff-based BP measurements, is presented in the tables of FIG. 11. As shown in FIG. 11, the mean absolute difference of the PPG-eBP is universally lower (better) than that of the volume clamp, both during the isometric leg press periods as well as the resting periods. It should be noted that, for each subject, a calibration period of both 5-minutes and 10-minutes was investigated, and a slight improvement in the PPG-BP model is observed for the longer calibration period (as can be derived from FIG. 11).

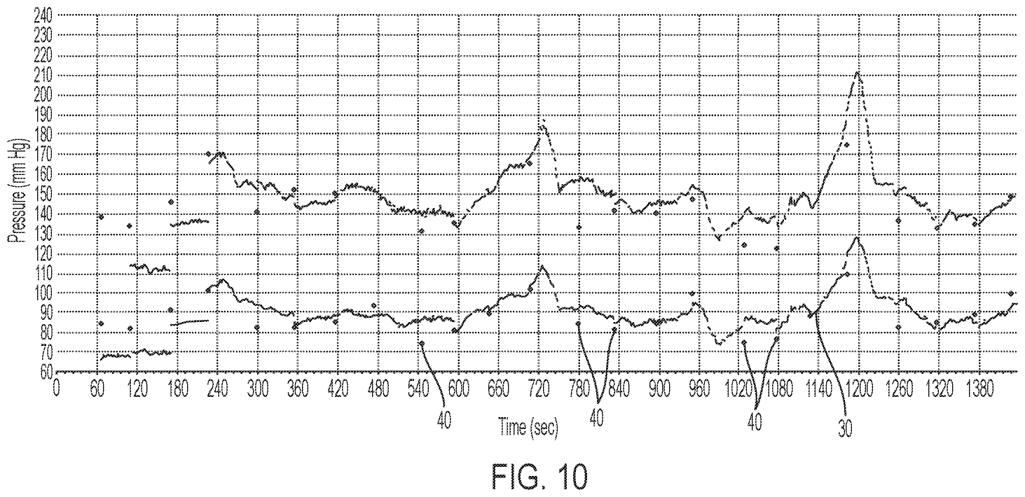

Referring to FIG. 10, BP estimates for a subject wearing a PPG sensor made over time via an adaptive predictive model in accordance with embodiments of the present invention are illustrated and represented by the plot 30. Actual blood pressure readings from a monitor attached to the subject are represented by the data points 40. BP estimation accuracy is improved over time as the adaptive predictive model is updated and this is illustrated in FIG. 10 as the distance between the plot 30 and the data points 40 decreases over time.

### Method of Generating a BP Estimation for a Subject Via an Adaptive Predictive Model

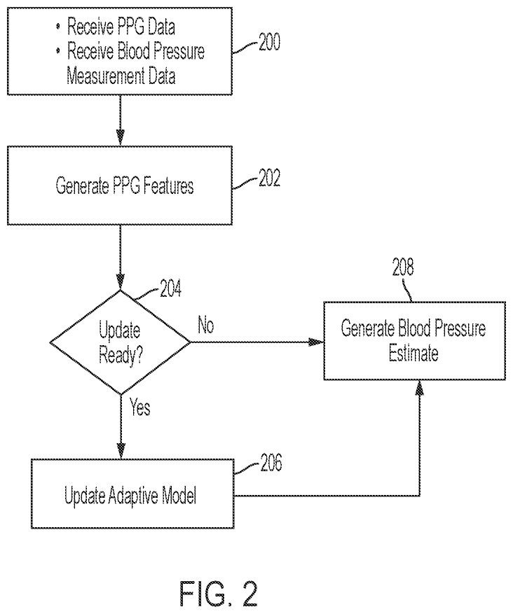

Referring to FIG. 2, a method of generating a BP estimation for a subject via a real-time adaptive predictive model executed via a computational system is illustrated. The method includes receiving, within a receiving period, real-time PPG data from a PPG sensor configured to measure PPG information from a subject, and receiving, within the receiving period, a real-time blood pressure measurement from a blood pressure monitoring device configured to measure the blood pressure of the subject (Block 200). Features are then generated from the received PPG data (Block 202). The generated features and the blood pressure measurement are stored in memory. If a blood pressure update from a monitoring device is ready (Block 204), the adaptive predictive model may be updated in real-time by processing the stored features and the stored blood pressure measurement to generate updated model parameters that reduce the estimation error of the adaptive predictive model (Block 206). A BP estimation for the subject is then generated via the updated adaptive predictive model (Block 208).

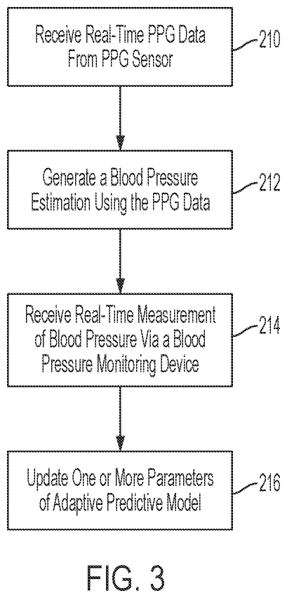

Referring to FIG. 3, a method of generating a BP estimation for a subject, according to some embodiments of the present invention, is illustrated. Real-time PPG data is received by a computational system (e.g., 100, FIG. 1) from a PPG sensor (e.g., 12-16, FIG. 5) attached to a subject (Block 210). The computational system generates a BP estimation for the subject via an adaptive predictive model using the PPG data (Block 212). A real-time measurement of blood pressure from a monitoring device (e.g., a blood pressure cuff 18, FIG. 5) attached to the subject is received by the computational system (Block 214) and the computational system updates one or more parameters of the adaptive predictive model (Block 216). This real-time blood pressure reading is used to adjust the adaptive predictive model such that the blood pressure estimation made by the adaptive predictive model using PPG data is closer to the actual blood pressure reading.

It is to be understood that the steps illustrated in FIG. 3 need not occur in the illustrated order. For example, real-time BP measurements (Block 214) may be collected prior to, or in unison with, the real-time PPG data collection (Block 210).

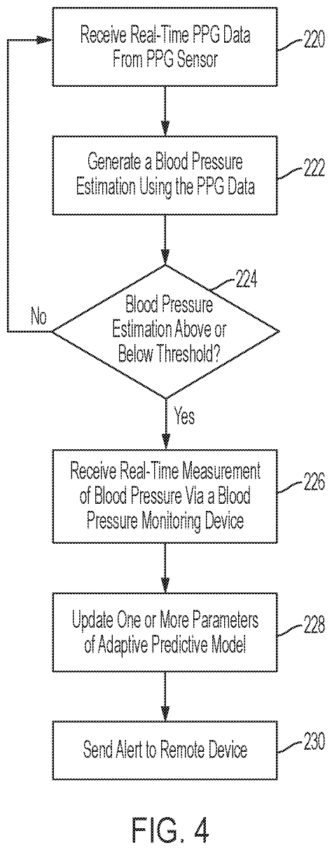

Referring to FIG. 4, a method of generating a BP estimation for a subject, according to some embodiments of the present invention, is illustrated. Real-time PPG data is received by a computational system (e.g., 100, FIG. 1) from a PPG sensor (e.g., 12-16, FIG. 5) attached to a subject (Block 220). The computational system generates a BP estimation for the subject via an adaptive predictive model using the PPG data (Block 222). A determination is made whether the BP estimation is above or below a threshold (Block 224). For example, a healthy blood pressure range is typically considered as systolic blood pressure less than 120 mmHg and diastolic less than 80 mmHg. However, if systolic blood pressure drops below 90 mmHg and/or diastolic blood pressure drops below 60 mmHg for a subject, medical intervention may be necessary. Similarly, if systolic blood pressure rises above 130 mmHg and/or diastolic blood pressure rises above 90 mmHg, medical intervention may be necessary.

If the BP estimation is above or below a threshold (Block 224), a real-time measurement of blood pressure is received by the computational system from a monitoring device (e.g., a blood pressure cuff 18, FIG. 5) attached to the subject (Block 226) and the computational system updates one or more parameters of the adaptive predictive model (Block 228). This real-time blood pressure reading is used to adjust the adaptive predictive model such that the blood pressure estimation made by the adaptive predictive model using PPG data is closer to the actual blood pressure reading. In addition, the computational system sends an alert to a remote device that the BP estimation is above or below a threshold (Block 230).

It should be noted that BP estimation does not have to fall outside of a range in order for a calibration cuff reading to be called for and then used to increase accuracy of the estimations. Estimated BP can be in a normal range and a subsequent cuff reading can still be used to refine the accuracy. The adaptive predictive model can be updated merely based on set timed cuff-based readings, without regard to BP values versus a threshold.

A remote device may be a smartphone of a medical provider, a nurse's station in a medical facility, or any other device that can alert a medical person as to the condition of the subject. The alert may also be sent to the blood pressure monitoring device (e.g., the blood pressure cuff 18, FIG. 5). In addition, the alert could be generated by the blood pressure monitoring device.

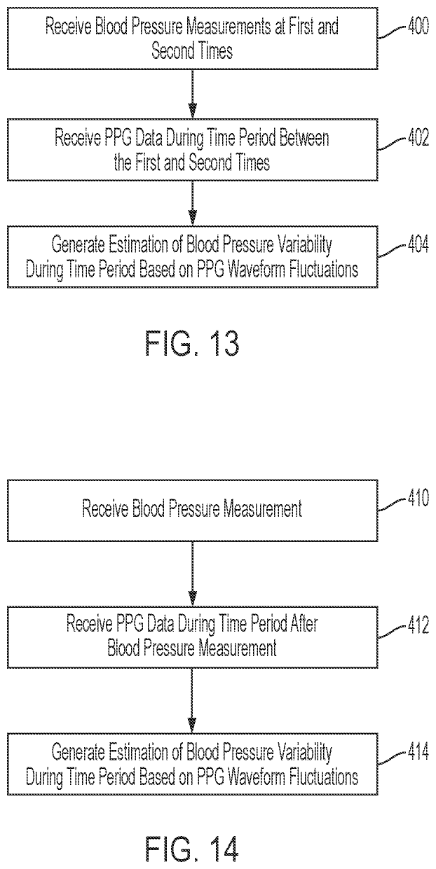

The methods illustrated in FIGS. 2-4 (and FIGS. 13-16 described below) may be executed via a computational system 100, such as that shown in FIG. 1. The computational system 100 may comprise: 1) at least one data bus 102 for receiving PPG data from a PPG sensor configured to measure PPG information from the subject and blood pressure data from a blood pressure monitoring device configured to measure a blood pressure of the subject, and 2) computational circuitry and instructions 104 configured to receive, within a receiving period, PPG data from the PPG sensor; receive, within the receiving period, a blood pressure measurement from the blood pressure monitoring device; generate features from the received PPG data; store the features in memory; store the blood pressure measurement in memory; update the current parameters of the adaptive predictive model by processing the stored features and the stored blood pressure measurement to generate updated model parameters that reduce the estimation error of the adaptive predictive model; and generate a BP estimation for the subject by executing the updated adaptive predictive model.

### Receive PPG Data; Receive BP Measurement

Referring back to FIG. 2, updating an adaptive predictive model (Block 206) requires at least two inputs: PPG features and at least one BP measurement. The data may be received over a “receiving period”, referring to a period of time wherein at least one PPG waveform and at least one time-correlated BP measurement has been received by the computational system 100 of FIG. 1. The received PPG data may be received as digitized data, and thus a prior digitization step may be required to digitally sample the PPG data (e.g., at a frequency of “fs”) before it is received by the computational system 100 of FIG. 1. The BP data may be received digitally as well, and thus a prior digitization step may also be required. However, discrete BP values may be received rather than streaming continuous BP values due to the discrete nature of cuff-based BP measurements. Although the PPG data and the BP measurement must be time-correlated (sufficiently close together in time), these measurements do not need to be exactly time-coincident. This is because BP may not change dramatically over the course of a few seconds in the vast majority of circumstances, and during these few seconds several PPG waveforms may be received. Moreover, because PPG data may be continually collected, whereas cuff-based BP measurements may require more than 60-to-90 seconds in between measurements, it may be impractical to perfectly align each PPG waveform with a coincident BP waveform. For a typical ambulatory resting state, a time-correlation between the PPG data and BP measurements within ˜30 seconds has been shown to be sufficient for continuous tracking. This timing may be longer or shorter depending on the activity status of the subject, the dynamics of the subject's cardiac output, or other factors that may affect the rate of BP changes or other physiological changes for the subject. This time-correlated PPG and BP measurement data may be stored in memory (such as a memory buffer) via the computational system.

### Generate PPG Features

The received PPG data is processed to generate a plurality of real-time PPG features (Block 202, FIG. 2). Each of these features may be a characteristic feature that is distinct from the other features, for a total of “n” characteristic features. Exemplary features include, but are not limited to time-domain features or transform-based features. Nonlimiting examples of time-domain features may comprise PPG amplitude, PPG upper and/or lower envelope, systolic and diastolic peak separation and/or relative amplitude, systolic and dicrotic notch peak-to-trough separation, temporal separations between key features (such as peaks or troughs) in a PPG waveform, and the like. Similarly, the PPG data may be processed to generate a derivative (e.g., a 1^{st}, 2^{nd}, 3^{rd}, etc. derivative) or an integral, and time-domain features of these derivative and/or integral waveforms may be generated (i.e., generating features for amplitude, relative amplitude of peaks or troughs, upper and/or lower envelope, temporal peak separations, and the like). Transform-based features may comprise spectral features, wavelet features, the Teager-Kaiser energy (KTE) operator based features, chirplet transform features, noiselet transform features, spaceogram features, shapelet features, derivative features, integral features, principle component analysis (PCA) features, and the like.

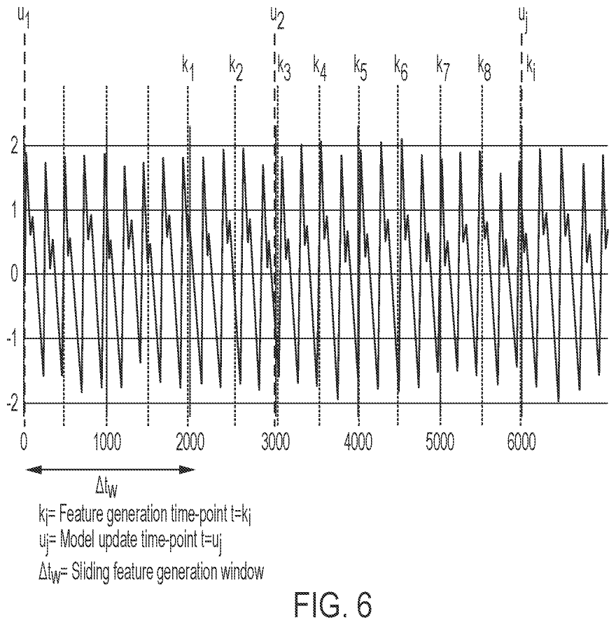

Generating features from the received PPG data may comprise generating features at feature generation intervals (time-points) t=kᵢ within the receiving period via a sliding window of time Δt_{w} (FIG. 6). Features may be generated at any point in time by the computational system; however, enough PPG data must be stored in memory in order to process a meaningful PPG feature—at least one full PPG wave, and preferably a plurality of PPG waveforms. For example, features may be generated at t=kᵢ over a feature generation window, by processing buffered digitized PPG data collected over a prior period of time that is Δt_{w} long (i.e., a window of time Δt_{w} wide). This feature generation window may comprise a sliding window, such as a FIFO (first-in-first-out) buffer, wherein the PPG data is stored in the buffer, continuously gaining a new sample of data, and losing the oldest sample of data over time. The feature generation process may comprise processing this buffered PPG in the time domain or via a transform of the stored time-domain data. It should be noted that a variety of different time-domain or transform-based processing methods may be utilized for generating the PPG features. Non-limiting examples of transforms for generating PPG features may comprise: spectral transforms, wavelet transforms, the Teager-Kaiser energy operator, chirplet transforms, noiselet transforms, spaceograms, shaplets, derivatives, integrals, and the like. Nonlimiting examples of time-domain processing may comprise processing steps for generating: PPG amplitude, PPG upper and/or lower envelope, systolic and diastolic peak separation, systolic and dicrotic notch peak-to-trough separation, and the like. Nonlimiting examples of transforms and time-domain processing that may be utilized are presented in U.S. Pat. No. 10,856,813 and PCT Application No. US20/49229, which are incorporated herein by reference in their entireties.

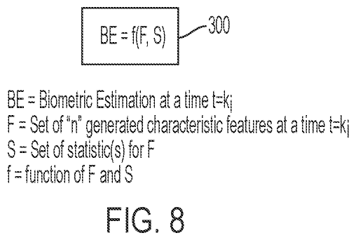

It should also be noted that, prior to generating a BP estimate, the PPG features (characteristic features) may be actively normalized (e.g., weighted), to help ensure smooth temporal tracking of PPG-based BP estimations (or other BP estimations) with BP measurements. One normalization approach is to process the statistics of the stored features (e.g., the prior stored PPG features in memory) and to normalize by these statistics. Normalization may be performed by processing historical data over a plurality of feature generation time-points, by generating statistics for the historical data and normalizing by these statistics. This normalization process may be updated with each new feature generation time point (e.g., t=kᵢ of FIG. 6 and FIG. 8). Alternatively, normalization may be performed with each model update (e.g., t=uⱼ of FIG. 6). There are numerous normalization methodologies known to those skilled in the art; a few examples may comprise: z-norming, min-max normalization, mean normalization, and the like. One nonlimiting normalization method to employ are Cochrane's equations for pooled statistics. To employ Cochrane's equations with each model update, the mean and standard deviation of each characteristic feature may be normalized (weighted) by processing (pooling) the statistics of the features from the past update (e.g., at t=uⱼ₋₁) with the statistics of the features following the past update (e.g., at t=uⱼ). Thus, the pooled mean and standard deviation generated by Cochrane's equations may be utilized as the basis for normalizing the characteristic features. As a specific non-limiting example, utilizing the z-norming method, the value of characteristic features may be normalized by the mean and standard deviation generated by Cochrane's equations—e.g., wherein this mean and standard deviation is generated by weighting the mean and standard deviation for the features from the past update (e.g., at t=uⱼ₋₁) with the mean and standard deviation of the features following the past update (e.g., at t=uⱼ).

The aforementioned feature statistics themselves may also be employed as features to an adaptive predictive model, according to embodiments of the present invention. This may help provide smoother tracking (e.g., of BP estimations vs. BP measurements).

It should be noted that, as part of (or prior to) feature generation, preprocessing of the received sensor information (e.g., the PPG sensor data) and/or the received BP measurement data (e.g., the BP measurement data) may be required. Additionally, it may be important to qualify the received data to reject “bad” data, generate a confidence score for the data, identify “good” data, or to classify data for further processing. A variety of preprocessing methodologies for PPG data (including associated motion sensor data) have been previously published and are well known to those skilled in the art, including, but not limited to: U.S. Pat. Nos. 10,834,483, 10,798,471, 10,631,740, 10,542,893, 10,512,403, 10,448,840, 9,993,204, 10,413,250, and PCT Application No. US20/49229, all of which are incorporated herein by reference in their entireties. Both passive and active methodologies of removing subject motion noise may be employed. Moreover, it should be noted that the optimal preprocessing may be feature-dependent. For example, regarding PPG data, for spectral domain features it may be desirable to remove or attenuate the “DC component” (e.g., the non-pulsatile component) from the PPG signal before feature generation. However, the DC component may be important for other features (such as time-domain features), or the DC component may even be a feature in itself. It should also be noted that PPG sensor data may comprise subject motion data (as described earlier), and this motion data may be utilized to reduce motion artifacts from optical sensor readings. The motion sensor may be integral to, or collocated with, the PPG sensor. Motion sensor data may be processed as a feature as well.

Preprocessing of BP measurement data may also be useful. For example, in a preferred use case, a BP measurement from a cuff-based BP monitor may comprise a discrete value of systolic and diastolic BP measurements. In some use cases, this data may be available to the computational system through an API (application programming interface) or through an application-specific interface. However, in some use cases, the BP measurement data received by the computational system of FIG. 1 may comprise a data stream (such as a raw data stream) where the BP measurement may need to be extracted via processing before the invention may be executed.

### Update Model Parameters

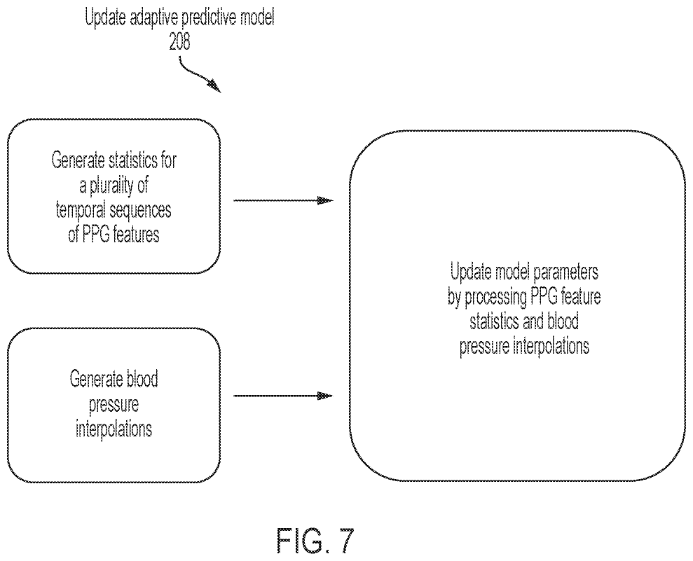

Referring to FIG. 8, an adaptive predictive model 300 for generating a BP estimation (BE) may take the form of BE=f(F,S), where F is a set of “n” generated characteristic features (e.g., normalized features) at a time t=kᵢ, and where S is a set of statistic(s) for F. The function f(F,S) may comprise a transfer function connecting the BP estimation with the aforementioned features and statistics. For each new BP measurement received, the adaptive predictive model 300 may be updated (as shown in FIG. 7) at each new update time-point t=uⱼ. Updating the model comprises updating one or more parameters of the adaptive predictive model 300.

Depending on the type of model used, the model parameters may be different. For example, in a regression model, the model parameters may comprise at least one coefficient to the regression model. Nonlimiting examples of suitable regression models may comprise: linear, SVM, random forest, neural network, decision trees, a combination of these models, and the like. Other types of models outside of regression models may also be utilized; as a nonlimiting example, a classifier may be utilized, or a combination of classification and regression (as may be utilized in a convolutional neural network (CNN)). Updating the model may comprise processing the characteristic features (e.g., normalized characteristic features) and a stored blood pressure measurement to generate updated model parameters that reduce the estimation error of the adaptive predictive model 300. For example, the regression model may be solved for the recent BP measurement and then the model coefficients may be updated. Alternatively, or additionally, a gradient-based optimization approach may be employed (such as classical gradient descent, Adam, Momentum, AdaGrad, RMSProp, AMSgrad, or the like) to update model coefficients with each new BP measurement.

Updating the adaptive predictive model in real-time may comprise processing a recent stored blood pressure measurement (associated with timepoint t=uⱼ) and a prior stored blood pressure measurement (associated with time-point t=uⱼ₋₁). In one embodiment, this may comprise generating an interpolation of expected blood pressure measurements (i.e., a temporal interpolation) between blood pressure measurements collected over time, such as an interpolation between the recent stored blood pressure measurement and the prior stored blood pressure measurement (or a plurality of prior stored blood pressure measurements). A specific example can be summarized in context of FIG. 6. A blood pressure measurement associated with time-point u₂ and a blood pressure measurement associated with time-point uⱼ (in this particular case u₃) may be stored in memory and processed to generate an interpolation of expected blood pressure measurements for plurality of feature generation intervals, such as for each feature generation interval t=kᵢ. In such case, updating the adaptive model may then comprise updating the model parameters in context of each feature set and each interpolated BP measurement over a plurality of intervals t=kᵢ. Thus, there is more information by which to optimize the regression model than just 2 blood pressure measurements, leading to smoother tracking of the BP estimation with the actual BP measurements.

### Generate BP Estimation

As summarized above, there are many model constructs that may be used to generate the BP estimation, and the general formalism of the function used to generate the BP estimation is presented in FIG. 8. For the specific case that has been described with respect to generating blood pressure estimations, the process of generating a BP estimation may comprise generating a systolic blood pressure, a diastolic blood pressure, a pulse pressure, a mean arterial pressure, or another type of pressure associated with blood flow. Moreover, the type of blood pressure that may be estimated may from virtually any location on the body, such as (but not limited to) brachial, thoracic, subclavian, femoral, tibial, radial, carotid, or the like. Each of these blood pressure estimations may be generated using the methods of FIGS. 2-4, via the processes summarized above; however, the BP measurement locations on the subject should ideally match that of the desired BP estimations. Namely, if the desired BP estimation comprises systolic and diastolic estimations of the brachial artery, then the BP monitoring device providing the BP measurements should (ideally) measure both the systolic and diastolic BP values from the brachial artery.

### Computational System for Generating a BP Estimation Via an Adaptive Predictive Model

For implementing the methods of FIGS. 2-4 and FIGS. 13-16, a computational system 100 may be utilized as shown in FIG. 1. The computational system 100 for generating a BP estimation for a subject via an adaptive predictive model may comprise: 1) at least one data bus 102 for receiving PPG data from a PPG sensor configured to measure PPG information from the subject and blood pressure data from a blood pressure monitoring device configured to measure a blood pressure of the subject, and 2) computational circuitry and instructions 104 configured to: a) receive, within a receiving period, PPG data from the PPG sensor, b) receive, within the receiving period, a blood pressure measurement from the blood pressure monitoring device, c) generate features from the received PPG data, d) store the features in memory, e) store the blood pressure measurement in memory, f) update the current parameters of the adaptive predictive model by processing the stored features and the stored blood pressure measurement to generate updated model parameters that reduce the estimation error of the adaptive predictive model, and g) generate a BP estimation for the subject by executing the updated adaptive predictive model.

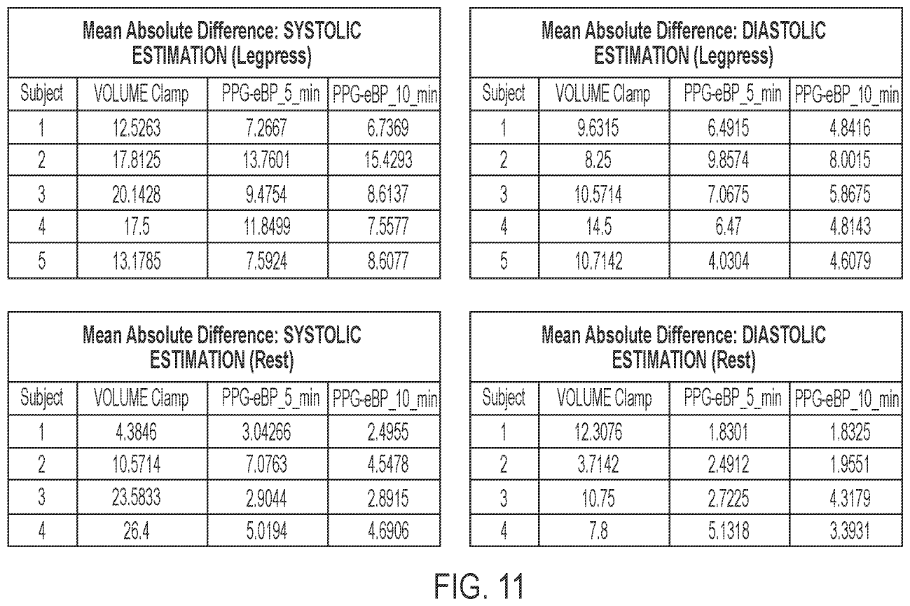

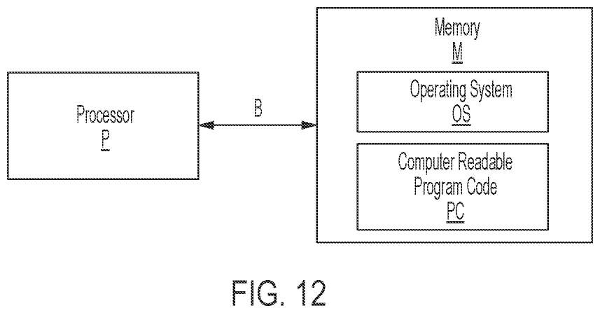

The computational system 100 may be implemented as a plurality of discrete components, a fully integrated system, or a mixture of both. For example, the computational system 100 may comprise a fully integrated microprocessor, with computational instructions for executing the processing steps of FIGS. 2-4 and FIGS. 13-16. FIG. 12 is a block diagram that illustrates details of an example processor P and memory M that may be used in accordance with various embodiments of the present invention. The processor P communicates with the memory M via an address/data bus B. The processor P may be, for example, a commercially available or custom microprocessor. Moreover, the processor P may include multiple processors. The memory M may be a non-transitory computer readable storage medium and may be representative of the overall hierarchy of memory/storage devices containing the software and data used to implement the methods of FIGS. 2-4 and FIGS. 13-16 as described herein. The memory M may include, but is not limited to, the following types of devices: cache, ROM, PROM, EPROM, EEPROM, flash, Static RAM (SRAM), and/or Dynamic RAM (DRAM).

The memory M may hold various categories of software and data, such as computer readable program code PC and/or an operating system OS. The operating system OS controls operations of the processor P, a PPG sensor (e.g., 12-16, FIG. 5), a BP monitoring device (e.g., cuff-based BP monitor 18, FIG. 5) and may coordinate execution of various programs by the processor P. For example, the computer readable program code PC, when executed by a processor P, may cause the processor P to perform any of the operations illustrated in the flowcharts of FIGS. 2-4 and FIGS. 13-16.

Alternatively, the computational system 100 may comprise an analog circuit configured to process the steps through analog processes. As another example, the computational instructions may be executed as a software library executed via a processor. As another example, the system may comprise neural circuitry. Both traditional or neural processors may be utilized, or a combination of both.

A variety of components for enabling the system 100 of FIG. 1 are well known to those skilled in the art. The computational resources required to execute the methods of FIGS. 2-4 and FIGS. 13-16 via a microprocessor are practical for a wearable or portable system, as the inventors have demonstrated via laboratory testing that suitable real-time performance can be achieved utilizing computational instructions (algorithms) executed via software on a commercially available smartphone 20 in communication with a wearable device 10-16, as illustrated in FIG. 5.

The system may comprise input/output lines (i.e., ports or buses) to communicate with other systems, for receiving and giving data from/to external systems. For example, the input/output lines may communicate with at least one external processor, computational system, or apparatus. In one specific embodiment, a BP estimation generated may be digitized and made available to an external computational system via a digital bus 106. In another embodiment, the input/output lines may communicate with one or more transceivers for communicating wirelessly with an external system. A variety of electronic communication configurations are well known to those skilled in the art.

In the case where a BP estimation is generated by the computational system of FIG. 1, for use by an external system, the external system may wish to send information to the computational system for modifying a computational process (i.e., modifying algorithms). For example, in one embodiment, the BP estimation generated may comprise a brachial BP estimation, where a remote system (in wired or wireless communication with the computational system) may comprise a cuff-based BP monitor that feeds BP measurements to the computational system of FIG. 1. The cuff-based BP monitor may also comprise a viewing screen to view PPG-BP estimation readings, generated by the computational system, in between BP measurements. It may be desirable to change the rate of PPG processing (such as the sampling rate, feature generation interval, update interval, or the like) via an interface on the cuff-based BP monitor, and this information may then be fed to the computational system of FIG. 1 as “external instruction data” for executing this desired change. Alternatively, or additionally, the computational system may have feedback to provide the external system (i.e., the cuff-based BP monitor), such as warnings that sensor estimations may be inaccurate due to motion noise, or other useful information.

It should be noted that one form of external system data may comprise meta data for the subject, and this meta data may be useful in processing BP estimations in accordance with embodiments of the present invention. Namely, the computational system 100 of FIG. 1 may receive external meta data (i.e., height, weight, age, sex, medication usages, and the like) for the subject and store this data in memory. The meta data may be utilized as a feature to the adaptive model 300 of FIG. 8. Alternatively, or additionally, this stored meta data may be utilized to create a profile for the subject. The profile may comprise parameters for the adaptive model that have been optimized for the subject (i.e., over the course of several BP measurements). A key benefit of a user profile is that it may prevent model adaption delays caused by a “cold start” (i.e., the subject starting a new estimation/measurement session). Phrased another way, a finite period of time may be required to adapt (calibrate) to the subject (as shown in FIG. 9), and this calibration phase can be shortened if the previous model parameters for the subject have been stored in memory.

### Generate Estimations of BP Variability

The variability of an individual's blood pressure is an important measure which can be used to predict other cardiovascular conditions. In the case of blood pressure variability (BPV) it is desirable to understand the fluctuations over time of how a person's systolic BP varies and how a person's diastolic BP varies. The difference may be that a person has a relatively normal measurement of systolic BP and diastolic BP at a given moment in time, but when BP is monitored from beat to beat, they may be experiencing large variations of systolic/diastolic above and below the momentary measure. Typically, BP is measured at intervals of 15 minutes or longer (although various other intervals may be utilized, also). BP variability can give valuable insight into what is happening to BP in between those momentary readings. Research indicates that higher blood pressure variability may be an indicator for other cardiovascular conditions and, thus, BPV has value in diagnosis.

According to the present invention, BP variability can be determined in various ways. For example, referring to FIG. 13, a blood pressure measurement for a subject can be obtained from a blood pressure monitoring device (e.g., an inflatable cuff configured to be attached to a limb or digit of a subject, etc.) at a first time, and another blood pressure measurement can be obtained at a second, later time (Block 400). PPG waveform data is acquired from a PPG sensor attached to the subject during the time period between the first time and the second time (Block 402). An estimation of blood pressure variability during the time period can be generated based on PPG waveform fluctuations identified during the time period (Block 404). The estimation of blood pressure variability may include an estimation of systolic blood pressure variation and/or an estimation of diastolic blood pressure variation. In some embodiments, the estimation of blood pressure variability may include an estimation of mean blood pressure variation.

Referring to FIG. 14, blood pressure variability may be estimated after a single blood pressure measurement. For example, a blood pressure measurement for a subject can be obtained (Block 410), and PPG waveform data can be obtained from the subject during a time period after obtaining the blood pressure measurement (Block 412). An estimation of blood pressure variability during the period of time after the blood pressure measurement can be generated based on PPG waveform fluctuations identified during the time period (Block 414).

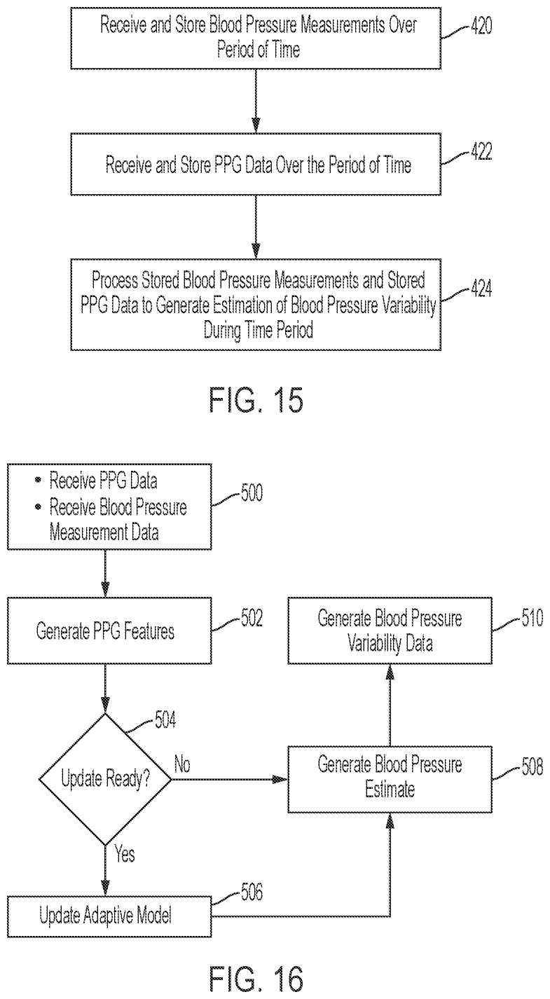

In addition, blood pressure variation can be determined using stored data. Referring to FIG. 15, blood pressure measurements from a blood pressure monitoring device attached to a subject can be received and stored over a period of time (Block 420). PPG data from a PPG sensor attached to the subject is also received and stored over the period of time (Block 422). The stored blood pressure measurements and the stored PPG data can then be processed to generate estimations of blood pressure variability during the time period (Block 424). In some embodiments, the PPG data includes PPG waveform data, and blood pressure variability is determined based on PPG waveform fluctuations identified during the time period.

Referring to FIG. 16, a method of generating a BP estimation for a subject via a real-time adaptive predictive model and also generating an estimation of BP variability is illustrated. The method includes receiving, within a receiving period, real-time PPG data from a PPG sensor configured to measure PPG information from a subject, and receiving, within the receiving period, real-time blood pressure measurement data from a blood pressure monitoring device configured to measure the blood pressure of the subject (Block 500). Features are then generated from the received PPG data (Block 502). The generated features and the blood pressure measurement are stored in memory. If a blood pressure update from the BP monitoring device is ready (Block 504), the adaptive predictive model may be updated in real-time by processing the stored features and the stored blood pressure measurement to generate updated model parameters that reduce the estimation error of the adaptive predictive model (Block 506), as described above. A BP estimation for the subject is then generated via the updated adaptive predictive model (Block 508). BPV data is then generated (Block 510).

BPV data can be generated (Block 510) in various ways. Blood pressure is typically measured as systolic and diastolic representing upper and lower measures of actual blood pressure in mmHg. In some embodiments, the systolic and diastolic blood pressure readings can be recorded for every heartbeat, and then a variation of those readings can be used to generate BP variability information.

In some embodiments, BPV data may be provided as an absolute measure such as “the standard deviation of your systolic BP was 12 mmHg during the measurement period”. In other embodiments, BPV may be represented as a relative index, or rating. It may be a dimensionless measure such as “your BPV index is +/−17%”. The measure may be based on variability of mean blood pressure (MAP), or variability of systolic, or variability of diastolic, or combinations of all three. However, even if BPV is represented as a dimensionless measure, the value provided is based off of a PPG sensor which is calibrated to periodic cuff measurements, as described above. BPV may be monitored and measured over long periods of time, for example weeks or months, to understand how a person's BPV responds to various treatments or medications, etc.

Example embodiments are described herein with reference to block diagrams and flow diagrams. It is understood that a block of the block diagrams and flow diagrams, and combinations of blocks in the block diagrams and flow diagrams, can be implemented by computer program instructions that are performed by one or more computer circuits, such as electrical circuits having analog and/or digital elements. These computer program instructions may be provided to a processor circuit of a general purpose computer circuit, special purpose computer circuit, and/or other programmable data processing circuit to produce a machine, such that the instructions, which execute via the processor of the computer and/or other programmable data processing apparatus, transform and control transistors, values stored in memory locations, and other hardware components within such circuitry to implement the functions/acts specified in the block diagrams and flow diagrams, and thereby create means (functionality) and/or structure for implementing the functions/acts specified in the block diagrams and flow diagrams.

These computer program instructions may also be stored in a tangible computer-readable medium that can direct a computer or other programmable data processing apparatus to function in a particular manner, such that the instructions stored in the computer-readable medium produce an article of manufacture including instructions which implement the functions/acts specified in the block diagrams and flow diagrams.

A tangible, non-transitory computer-readable medium may include an electronic, magnetic, optical, electromagnetic, or semiconductor data storage system, apparatus, or device. More specific examples of the computer-readable medium would include the following: a portable computer diskette, a random access memory (RAM) circuit, a read-only memory (ROM) circuit, an erasable programmable read-only memory (EPROM or Flash memory) circuit, a portable compact disc read-only memory (CD-ROM), and a portable digital video disc read-only memory (DVD/BlueRay).

The computer program instructions may also be loaded onto a computer and/or other programmable data processing apparatus to cause a series of operational steps to be performed on the computer and/or other programmable apparatus to produce a computer-implemented process such that the instructions which execute on the computer or other programmable apparatus provide steps for implementing the functions/acts specified in the block diagrams and flow diagrams. Accordingly, embodiments of the present invention may be embodied in hardware and/or in software (including firmware, resident software, micro-code, etc.) that runs on a processor such as a digital signal processor, which may collectively be referred to as “logic”, “circuitry”, “a module”, “an engine” or variants thereof.

It should also be noted that the functionality of a given block of the block diagrams and flow diagrams may be separated into multiple blocks and/or the functionality of two or more blocks of the block diagrams and flow diagrams may be at least partially integrated. Finally, other blocks may be added/inserted between the blocks that are illustrated. Moreover, although some of the diagrams include arrows on communication paths to show a primary direction of communication, it is to be understood that communication may occur in the opposite direction to the depicted arrows.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

1. A blood pressure monitoring system, comprising:
a blood pressure monitoring device configured to obtain a blood pressure measurement from a subject;
an arterial pulse wave sensor configured to obtain arterial pulse wave data from the subject; and
at least one processor configured to:
generate a blood pressure estimation for the subject via an adaptive predictive model using real-time arterial pulse wave data from the arterial pulse wave sensor;
receive a real-time blood pressure measurement from the blood pressure monitoring device; and
in response to receiving the real-time blood pressure measurement, update one or more parameters of the adaptive predictive model in real-time to improve blood pressure estimation accuracy of the adaptive predictive model.

2. The system of claim 1, wherein the at least one processor is further configured to:
determine whether the generated blood pressure estimation is above or below one or more thresholds; and
in response to determining that the generated blood pressure estimation is above or below the one or more thresholds, update the one or more parameters of the adaptive predictive model in real-time.

3. The system of claim 1, wherein the at least one processor is further configured to, in response to receiving one or more subsequent real-time blood pressure measurements, update the one or more parameters of the adaptive predictive model in real-time.

4. The system of claim 1, wherein the at least one processor is further configured to send an alert to a remote device in response to determining that the generated blood pressure estimation is above or below a threshold.

5. The system of claim 2, wherein the at least one processor is further configured to request a blood pressure measurement from the blood pressure monitoring device in response to determining that the generated blood pressure estimation is above or below the one or more thresholds.

6. The system of claim 1, wherein the adaptive predictive model comprises one of a regression model, a machine learning model, or a classifier model.

7. The system of claim 1, wherein the arterial pulse wave sensor comprises a photoplethysmography (PPG) sensor.

8. The system of claim 1, wherein the blood pressure monitoring device comprises an inflatable cuff configured to be attached to a limb or digit of a subject.

9. A wearable device, comprising:
an automated inflatable cuff configured to be attached to a limb or digit of a subject, wherein the cuff is configured to generate a blood pressure measurement for the subject;
an arterial pulse wave sensor configured to obtain arterial pulse wave data from the subject; and
at least one processor configured to:
generate a blood pressure estimation for the subject via an adaptive predictive model using real-time arterial pulse wave data from the arterial pulse wave sensor;
receive a real-time blood pressure measurement from the cuff, and
in response to receiving the real-time blood pressure measurement, update one or more parameters of the adaptive predictive model in real-time to improve blood pressure estimation accuracy of the adaptive predictive model.

10. The wearable device of claim 9, wherein the at least one processor is further configured to:
determine whether the generated blood pressure estimation is above or below one or more thresholds; and
in response to determining that the generated blood pressure estimation is above or below the one or more thresholds, update the one or more parameters of the adaptive predictive model in real-time.

11. The wearable device of claim 9, wherein the at least one processor is further configured to, in response to receiving one or more subsequent real-time blood pressure measurements, update the one or more parameters of the adaptive predictive model in real-time.

12. The wearable device of claim 9, wherein the at least one processor is further configured to send an alert to a remote device in response to determining that the generated blood pressure estimation is above or below a threshold.

13. The wearable device of claim 10, wherein the at least one processor is further configured to request a blood pressure measurement from the cuff in response to determining that the generated blood pressure estimation is above or below the one or more thresholds.

14. The wearable device of claim 9, wherein the adaptive predictive model comprises one of a regression model, a machine learning model, or a classifier model.

15. The wearable device of claim 9, wherein the arterial pulse wave sensor comprises a photoplethysmography (PPG) sensor.

16-38. (canceled)

39. A blood pressure monitoring method comprising the following steps performed by at least one processor:
generating a blood pressure estimation for a subject via an adaptive predictive model using real-time arterial pulse wave data from an arterial pulse wave sensor attached to the subject;
receiving a real-time blood pressure measurement from a blood pressure monitoring device attached to the subject; and
in response to receiving the real-time blood pressure measurement, updating one or more parameters of the adaptive predictive model in real-time to improve blood pressure estimation accuracy of the adaptive predictive model.

40. The method of claim 39, wherein the arterial pulse wave sensor comprises a photoplethysmography (PPG) sensor, and wherein the arterial pulse wave data comprises PPG waveform data.

41. The method of claim 39, wherein the blood pressure monitoring device comprises an inflatable cuff configured to be attached to the limb or digit of a subject.

42. The method of claim 39, further comprising the following steps performed by the at least one processor:
determining whether the generated blood pressure estimation is above or below one or more thresholds; and
in response to determining that the generated blood pressure estimation is above or below the one or more thresholds, updating the one or more parameters of the adaptive predictive model in real-time.

43. The method of claim 39, further comprising the following step performed by the at least one processor:
in response to receiving one or more subsequent real-time blood pressure measurements, updating the one or more parameters of the adaptive predictive model in real-time.
